(19) United States
(12) Patent Application Publication
(10) Pub. No.: US 2026/0327634 A1
(43) Pub. Date: Oct. 22, 2026

(54) METHODS AND MICROORGANISMS FOR PRODUCTION OF A PRODUCT USING ETHANOL AS A CARBON SOURCE

(71) Applicant: DMC BIOTECHNOLOGIES, INC., Boulder, CO (US)

(72) Inventors: Taylor LUNDY, Boulder, CO (US); Zhixia YE, Boulder, CO (US)

(21) Appl. No.: 19/486,077

(22) PCT Filed: May 22, 2024
(86) PCT No.: PCT/US2024/030452

§ 371 Date: Nov. 20, 2025

Related U.S. Application Data
(60) Provisional application No. 63/503,527, filed on May 22, 2023.

(51) Int. Cl. C12N 15/70; C12N 1/20; C12N 15/52; C12P 7/22; C12P 7/44

(52) U.S. Cl. CPC C12N 15/70; C12N 1/20; C12N 15/52; C12P 7/22; C12P 7/44

(57) ABSTRACT

Methods and microorganisms for production of a product using ethanol as a carbon source are provided. A multi-stage biofermentation process in which a genetically modified microorganism adapated to grow in a growth media comprising ethanol is provided. The growth media may comprise no added glucose.

### CROSS-REFERENCE TO RELATED APPLICATIONS

This application is a national phate entry of PCT/US24/30452, filed May 22, 2024, which claims the benefit of priority to U.S. provisional patent application No. 63/503,527, filed on May 23, 2023, which is incorporated by reference herein in its entirety.

### BACKGROUND

Through evolution, microbiological organisms have developed many strategies to adapt to their environments, including by using different carbon sources. For a microorgansism that has been genetically modified so as to optimally produce a particular product, flexibility in using more than one type of carbon source is an attractive feature. This is especially true where at least one of the alternative carbon sources is inexpensive and abundantly available, such as ethanol.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, a multi-stage biofermentation process for producing a product from a genetically modified microorganism is provided, the process comprising: (A) providing a genetically modified microorganism that: (1) is adapted to grow in a growth media comprising ethanol; and (2) comprises: (a) a production pathway comprising at least one production enzyme for biosynthesis of the product; (b) a mutation of the endogenous adhE gene and of the endogenous adhE gene promotor; and (c) one or more synthetic metabolic valves for reducing or eliminating flux through multiple metabolic pathways within the genetically modified microorganism when the synthetic metabolic valves are induced; (B) growing the genetically modified microorganism in a media comprising ethanol in a growth phase; and (C) transitioning to a productive stationary phase, the transitioning comprising: (1) depleting a limiting nutrient; (2) inducing the one or more synthetic metabolic valves; and (3) activating the production pathway.

In other aspect, a method is provided for adapting a genetically modified E. coli microorganism comprising synthetic metabolic valves to grow aerobically in a growth medium comprising ethanol, the method comprising: providing a microorganism with a deletion of endogenous adhE gene; growing the microorganism in an ethanol minimal media; modifying the microorgansism with plasmids encoding synthetic metabolic valves; and propagating the modified microorganism in a growth process in a media comprising SM10++ or FGM10.2_SF 1 g/L glucose and 10 g/L ethanol for at least three propagations.

In another aspect, a genetically modified E. coli microorganism comprising a production pathway is provided, the microorganism comprising: at least one production enzyme for biosynthesis of the product; a mutation of the endogenous adhE gene and of the endogenous adhE gene promotor; and one or more synthetic metabolic valves for reducing or eliminating flux through multiple metabolic pathways within the genetically modified microorganism when the synthetic metabolic valves are induced.

Other methods, features, and advantages are, or will become, apparent upon examination of the following figures and Detailed Description. All such additional methods, features, and advantages are intended to be included within this description and are protected by the accompanying claims.

### BRIEF DESCRIPTION OF THE SEQUENCES

EM7 promoter:
(SEQ ID NO: 1)
GGTTTAGTTCCTCACCTTGTCGTATTATACTATGCCGATATACTATGCCG
ATGATTAATTGTCAAC
EM7* promoter:
(SEQ ID NO: 2)
GTTGACAATTAATCATCGGCATAGTATAATACGAC

adhE A267T/E568K mutant gene (first reported in PMID: 10922373)-changes to the nucleotide sequence are underlined, italicized, and written in lower case:

(SEQ ID NO: 3)
ATGGCTGTTACTAATGTCGCTGAACTTAACGCACTCGTAGAGCGTGTAAAAAAA
GCCCAGCGTGAATATGCCAGTTTCACTCAAGAGCAAGTAGACAAAATCTTCCGCGCCGCCGCTCT
GGCTGCTGCAGATGCTCGAATCCCACTCGCGAAAATGGCCGTTGCCGAATCCGGCATGGGTATCG
TCGAAGATAAAGTGATCAAAAACCACTTTGCTTCTGAATATATCTACAACGCCTATAAAGATGA
AAAAACCTGTGGTGTTCTGTCTGAAGACGACACTTTTGGTACCATCACTATCGCTGAACCAATCG
GTATTATTTGCGGTATCGTTCCGACCACTAACCCGACTTCAACTGCTATCTTCAAATCGCTGATC
AGTCTGAAGACCCGTAACGCCATTATCTTCTCCCCGCACCCGCGTGCAAAAGATGCCACCAACAA
AGCGGCTGATATCGTTCTGCAGGCTGCTATCGCTGCCGGTGCTCCGAAAGATCTGATCGGCTGGA
TCGATCAACCTTCTGTTGAACTGTCTAACGCACTGATGCACCACCCAGACATCAACCTGATCCTC
GCGACTGGTGGTCCGGGCATGGTTAAAGCCGCATACAGCTCCGGTAAACCAGCTATCGGTGTAGG
CGCGGGCAACACTCCAGTTGTTATCGATGAAACTGCTGATATCAAACGTGCAGTTGCATCTGTAC
TGATGTCCAAAACCTTCGACAACGGCGTAATCTGTGCTTCTGAACAGTCTGTTGTTGTTGTTGAC
TCTGTTTATGACGCTGTACGTGAACGTTTTaccACCCACGGCGGCTATCTGTTGCAGGGTAAAGA
GCTGAAAGCTGTTCAGGATGTTATCCTGAAAAACGGTGCGCTGAACGCGGCTATCGTTGGTCAGC
CAGCCTATAAAATTGCTGAACTGGCAGGCTTCTCTGTACCAGAAAACACCAAGATTCTGATCGGT
GAAGTGACCGTTGTTGATGAAAGCGAACCGTTCGCACATGAAAAACTGTCCCCGACTCTGGCAAT
GTACCGCGCTAAAGATTTCGAAGACGCGGTAGAAAAAGCAGAGAAACTGGTTGCTATGGGCGGT
ATCGGTCATACCTCTTGCCTGTACACTGACCAGGATAACCAACCGGCTCGCGTTTCTTACTTCGG
TCAGAAAATGAAAACGGCGCGTATCCTGATTAACACCCCAGCGTCTCAGGGTGGTATCGGTGACC
TGTATAACTTCAAACTCGCACCTTCCCTGACTCTGGGTTGTGGTTCTTGGGGTGGTAACTCCATC
TCTGAAAACGTTGGTCCGAAACACCTGATCAACAAGAAAACCGTTGCTAAGCGAGCTGAAAACA
TGTTGTGGCACAAACTTCCGAAATCTATCTACTTCCGCCGTGGCTCCCTGCCAATCGCGCTGGAT
GAAGTGATTACTGATGGCCACAAACGTGCGCTCATCGTGACTGACCGCTTCCTGTTCAACAATGG
TTATGCTGATCAGATCACTTCCGTACTGAAAGCAGCAGGCGTTGAAACTGAAGTCTTCTTCGAAG
TAGAAGCGGACCCGACCCTGAGCATCGTTCGTAAAGGTGCAGAACTGGCAAACTCCTTCAAACCA
GACGTGATTATCGCGCTGGGTGGTGGTTCCCCGATGGACGCCGCGAAGATCATGTGGGTTATGTA
CGAACATCCGGAAACTCACTTCGAAaaaCTGGCGCTGCGCTTTATGGATATCCGTAAACGTATCT
ACAAGTTCCCGAAAATGGGCGTGAAAGCGAAAATGATCGCTGTCACCACCACTTCTGGTACAGGT
TCTGAAGTCACTCCGTTTGCGGTTGTAACTGACGACGCTACTGGTCAGAAATATCCGCTGGCAGA
CTATGCGCTGACTCCGGATATGGCGATTGTCGACGCCAACCTGGTTATGGACATGCCGAAGTCCC
TGTGTGCTTTCGGTGGTCTGGACGCAGTAACTCACGCCATGGAAGCTTATGTTTCTGTACTGGCA
TCTGAGTTCTCTGATGGTCAGGCTCTGCAGGCACTGAAACTGCTGAAAGAATATCTGCCAGCGTC
CTACCACGAAGGGTCTAAAAATCCGGTAGCGCGTGAACGTGTTCACAGTGCAGCGACTATCGCGG
GTATCGCGTTTGCGAACGCCTTCCTGGGTGTATGTCACTCAATGGCGCACAAACTGGGTTCCCAG
TTCCATATTCCGCACGGTCTGGCAAACGCCCTGCTGATTTGTAACGTTATTCGCTACAATGCGAA
CGACAACCCGACCAAGCAGACTGCATTCAGCCAGTATGACCGTCCGCAGGCTCGCCGTCGTTATG
CTGAAATTGCCGACCACTTGGGTCTGAGCGCACCGGGCGACCGTACTGCTGCTAAGATCGAGAAA
CTGCTGGCATGGCTGGAAACGCTGAAAGCTGAACTGGGTATTCCGAAATCTATCCGTGAAGCTGG
CGTTCAGGAAGCAGACTTCCTGGCGAACGTGGATAAACTGTCTGAAGATGCATTCGATGACCAGT
GCACCGGCGCTAACCCGCGTTACCCGCTGATCTCCGAGCTGAAACAGATTCTGCTGGATACCTAC
TACGGTCGTGATTATGTAGAAGGTGAAACTGCAGCGAAGAAAGAAGCTGCTCCGGCTAAAGCTG
AGAAAAAAGCGAAAAAATCCGCTTAA

AdhE A267T/E568K mutant polypeptide-amino acid substitutions are underlined and italicized:

(SEQ ID NO: 4)
MAVTNVAELNALVERVKKAQREYASFTQEQVDKIFRAAALAAADARIPLAKMAV
AESGMGIVEDKVIKNHFASEYIYNAYKDEKTCGVLSEDDTFGTITIAEPIGIICGIVPTTNPTSTAIFK
SLISLKTRNAIIFSPHPRAKDATNKAADIVLQAAIAAGAPKDLIGWIDQPSVELSNALMHHPDINLI
LATGGPGMVKAAYSSGKPAIGVGAGNTPVVIDETADIKRAVASVLMSKTFDNGVICASEQSVVVVD
SVYDAVRERFTTHGGYLLQGKELKAVQDVILKNGALNAAIVGQPAYKIAELAGFSVPENTKILIGEV
TVVDESEPFAHEKLSPTLAMYRAKDFEDAVEKAEKLVAMGGIGHTSCLYTDQDNQPARVSYFGQ
KMKTARILINTPASQGGIGDLYNFKLAPSLTLGCGSWGGNSISENVGPKHLINKKTVAKRAENML
WHKLPKSIYFRRGSLPIALDEVITDGHKRALIVTDRFLFNNGYADQITSVLKAAGVETEVFFEVEAD
PTLSIVRKGAELANSFKPDVIIALGGGSPMDAAKIMWVMYEHPETHFEKLALRFMDIRKRIYKFPK
MGVKAKMIAVTTTSGTGSEVTPFAVVTDDATGQKYPLADYALTPDMAIVDANLVMDMPKSLCAF
GGLDAVTHAMEAYVSVLASEFSDGQALQALKLLKEYLPASYHEGSKNPVARERVHSAATIAGIAFA
NAFLGVCHSMAHKLGSQFHIPHGLANALLICNVIRYNANDNPTKQTAFSQYDRPQARRRYAEIAD
HLGLSAPGDRTAAKIEKLLAWLETLKAELGIPKSIREAGVQEADFLANVDKLSEDAFDDQCTGANP
RYPLISELKQILLDTYYGRDYVEGETAAKKEAAPAKAEKKAKKSA

### BRIEF DESCRIPTION OF THE FIGURES

The novel features of the invention are set forth with particularity in the claims. A better understanding of the features and advantages of the present invention will be obtained by reference to the following Detailed Description that sets forth illustrative aspects, in which the principles of the invention are used, and the accompanying drawings of which:

FIG. 1 depicts a bar graph of growth of bacterial cells (represented by cell density (OD600)) versus time in ethanol minimal media in a microorganism expressing the adhE A267T/E568K mutant gene which encodes for the adhE A267T/E568K polypeptide mutant, as compared to controls.

FIG. 2 depicts an ethanol only adaptation growth process.

FIG. 3 compares the kinetics of growth of bacterial cells (represented by cell density (OD600)): (A) in ethanol, where the endogenous chromosomal adhE gene has been deleted from the cells, and (1) the cells have been transformed by a plasmid based adhE A267T/E568K mutant gene; or (2) the endogenous adhE gene has been replaced with a chromosome based adhE A267T/E568K mutant gene; or (B) in glucose, using the endogenous adhE gene.

FIG. 4 depicts a graph representing shake flask production of a product from ethanol.

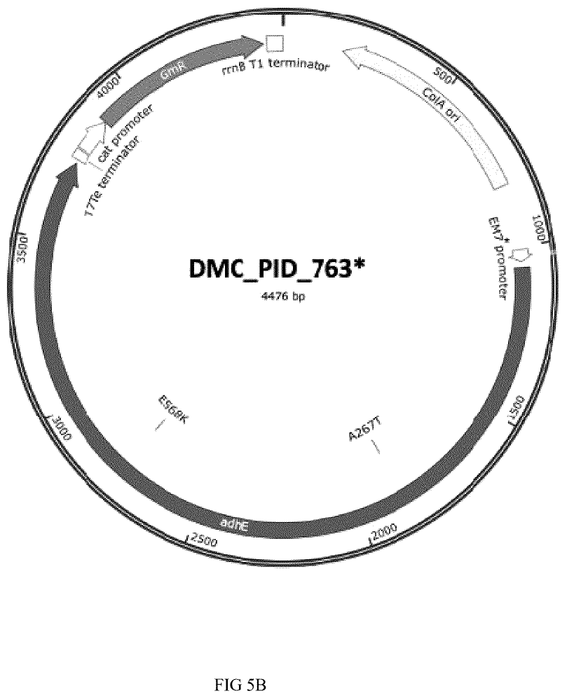

FIG. 5 depicts plasmid and genome maps for transformation or deletion and replacement as described with respect to FIG. 3, respectively, of adhE A267T/E568K mutant gene into E. coli.

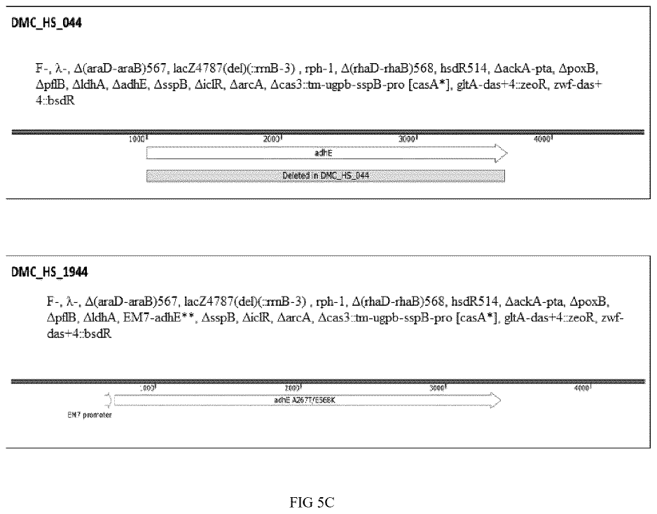

### DETAILED DESCRIPTION

### I. Definitions

Unless otherwise defined, all technical and scientific terms used herein have the same meaning as commonly understood by one of ordinary skill in the art to which this invention pertains. In case of conflict, the present specification, including definitions, will control.

Unless otherwise specified, “a,” “an,” “the,” “one or more of,” and “at least one” are used interchangeably. The singular forms “a”, “an,” and “the” are inclusive of their plural forms.

The recitations of numerical ranges by endpoints include all numbers subsumed within that range (e.g., 0.5 to 5 includes 1, 1.5, 2, 2.75, 3, 3.80, 4, 5, etc.).

The term “about,” when referring to a value or to an amount of mass, weight, time, volume, concentration, or percentage, is meant to encompass variations of +10% from the specified amount. The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

Moreover, the present disclosure also contemplates that in some aspects, any feature or combination of features set forth herein can be excluded or omitted. To illustrate, if the specification states that a complex comprises components A, B, and C, it is specifically reserved that any of A, B, or C, or a combination thereof, can be omitted and disclaimed singularly or in any combination.

The term “amino acid modification” includes an amino acid substitution, insertion, or deletion in a polypeptide sequence. By “amino acid substitution” or “substitution” is meant the replacement of an amino acid at a particular position in a parent polypeptide sequence with another amino acid. For example, the substitution A267T refers to a modified polypeptide in which the alanine at position 267 is replaced with a threonine. Multiple substitutions are typically separated by a slash or a comma. For example, A267T/E568K refers to a double variant comprising the substitutions A267T and E568K. By “amino acid insertion” or “insertion” is meant the addition of an amino acid at a particular position in a parent polypeptide sequence. For example, insert-267 designates an insertion at position 267. By “amino acid deletion” or “deletion” is meant the removal of an amino acid at a particular position in a parent polypeptide sequence. For example, A267-designates the deletion of alanine at position 267.

The term “heterologous DNA,” “heterologous nucleic acid sequence,” and the like as used herein refer to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid, such as a nonnative promoter driving gene expression. The term “heterologous” is intended to include the term “exogenous” as the latter term is generally used in the art. With reference to the host microorganism's genome prior to the introduction of a heterologous nucleic acid sequence, the nucleic acid sequence that codes for the enzyme is heterologous (whether or not the heterologous nucleic acid sequence is introduced into that genome). As used herein, “chromosomal” and “native” and “endogenous” refer to genetic material of the host microorganism.

As used herein, the term “gene disruption” or grammatical equivalents thereof (and including “to disrupt enzymatic function,” “disruption of enzymatic function,” and the like) is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduce activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

Bio-production, Micro-fermentation (microfermentation), or Fermentation, as used herein, may be aerobic, microaerobic, or anaerobic.

When the genetic modification of a gene product, e.g., an enzyme, is referred to herein, including the claims, the genetic modification is of a nucleic acid sequence, such as or including the gene, that normally encodes the stated gene product, i.e., the enzyme.

Species and other phylogenic identifications are according to the classification known to a person skilled in the art of microbiology.

Enzymes are listed here within, with reference to a UniProt identification number, which would be well known to one skilled in the art. The UniProt database can be accessed at www.UniProt.org. When the genetic modification of a gene product, e.g., an enzyme, is referred to herein, including in the claims, the genetic modification is of a nucleic acid sequence, such as or including the gene, that normally encodes the stated gene product, i.e., the enzyme.

Where methods and steps described herein indicate certain events occurring in certain order, those of ordinary skill in the art will recognize that the ordering of certain steps may be modified, and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, “DCW” means dry cell weight, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “OD” means optical density, “OD600” means the optical density measured at a photon wavelength of 600 nm, “kDa” means kilodaltons, “g” means the gravitation constant, “bp” means base pair(s), “kbp” means kilobase pair(s), “% w/v” means weight/volume percent, “% v/v” means volume/volume percent, “IPTG” means isopropyl-μ-D-thiogalactopyranoiside, “aTc” means anhydrotetracycline, “RBS” means ribosome binding site, “rpm” means revolutions per minute, “HPLC” means high performance liquid chromatography, and “GC” means gas chromatography.

While various aspects of the present invention have been shown and described herein, it is emphasized that such aspects are provided by way of example only. Numerous variations, changes, and substitutions may be made without departing from the invention herein in its various aspects. Specifically, and for whatever reason, for any grouping of compounds, nucleic acid sequences, polypeptides, including specific proteins such as functional enzymes, metabolic pathway enzymes or intermediates, elements, or other compositions, or concentrations stated or otherwise presented herein in a list, table, or other grouping unless clearly stated otherwise, each such grouping provides the basis for and serves to identify various subset aspects, the subset aspects in their broadest scope comprising every subset of such grouping by exclusion of one or more members (or subsets) of the respective stated grouping. Moreover, when any range is described herein, unless clearly stated otherwise, that range includes all values therein and all sub-ranges therein.

### General Consideration

### II. Microorganisms

Features as described and claimed herein may be provided in a microorganism selected from the listing herein, or another suitable microorganism, that also comprises one or more natural, introduced, or enhanced product bio-production pathways. Thus, in some aspects, the microorganism(s) comprises an endogenous product production pathway (which may, in some such aspects, be enhanced), whereas in other aspects the microorganism does not comprise an endogenous product production pathway.

More particularly, based on the various criteria described herein, suitable microbial hosts for the bio-production of a chemical product generally may include, but are not limited to the organisms described in the Methods Section.

The host microorganism or the source microorganism for any gene or protein described herein may be selected from the following list of microorganisms: Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia, Salmonella, Bacillus, Streptomyces, and Pseudomonas. In some aspects the host microorganism is an E. coli microorganism.

### III. Bio-Production Reactors and Systems

Fermentation systems utilizing methods and/or compositions according to the invention are also within the scope of the invention. Any of the recombinant microorganisms as described and/or referred to herein may be introduced into an industrial bio-production system where the microorganisms convert a carbon source into a product in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to a selected chemical product. Bio-productions may be performed under aerobic, microaerobic, or anaerobic conditions, with or without agitation. Industrial bio-production systems and their operation are well-known to those skilled in the arts of chemical engineering and bioprocess engineering.

The amount of a product produced in a bio-production media generally can be determined using a number of methods known in the art, for example, high performance liquid chromatography (HPLC), gas chromatography (GC), or GC/Mass Spectroscopy (MS).

### IV. Genetic Modifications, Nucleotide Sequences, and Amino Acid Sequences

Aspects of the present invention may result from introduction of an expression vector into a host microorganism, wherein the expression vector contains a nucleic acid sequence coding for an enzyme that is, or is not, normally found in a host microorganism.

The ability to genetically modify a host cell is essential for the production of any genetically modified (recombinant) microorganism. The mode of gene transfer technology may be by electroporation, conjugation, transduction, or natural transformation. A broad range of host conjugative plasmids and drug resistance markers are available. The cloning vectors are tailored to the host organisms based on the nature of antibiotic resistance markers that can function in that host. Also, as disclosed herein, a genetically modified (recombinant) microorganism may comprise modifications other than via plasmid introduction, including modifications to its genomic DNA.

More generally, nucleic acid constructs can be prepared comprising an isolated polynucleotide encoding a polypeptide having enzyme activity operably linked to one or more (several) control sequences that direct the expression of the coding sequence in a microorganism, such as E. coli, under conditions compatible with the control sequences.

The isolated polynucleotide may be manipulated to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well established in the art.

The control sequence may be an appropriate promoter sequence, a nucleotide sequence that is recognized by a host cell for expression of a polynucleotide encoding a polypeptide of the present invention. The promoter sequence may contain transcriptional control sequences that mediate the expression of the polypeptide. The promoter may be any nucleotide sequence that shows transcriptional activity in the host cell of choice, including mutant, truncated, and hybrid promoters, and may be obtained from genes encoding extracellular or intracellular polypeptides either homologous or heterologous to the host cell. The techniques for modifying and using recombinant DNA promoter sequences are well established in the art.

For various aspects of the invention, the genetic manipulations may include a manipulation directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected culture conditions. Genetic manipulation of nucleic acid sequences may increase copy number and/or comprise use of mutants of an enzyme related to product production. Specific methodologies and approaches to achieve such genetic modification are well known to one skilled in the art.

In various aspects, to function more efficiently, a microorganism may comprise one or more gene deletions. For example, in E. coli, the genes encoding the lactate dehydrogenase (ldhA), phosphate acetyltransferase (pta), pyruvate oxidase (poxB), pyruvate-formate lyase (pflB), methylglyoxal synthase (mgsA), acetate kinase (ackA), clpXP protease specificity enhancing factor (sspB), ATP-dependent Lon protease (lon), outer membrane protease (ompT), arcA transcriptional dual regulator (arcA), and iclR transcriptional regulator (iclR) may be disrupted, including deleted. Such gene disruptions, including deletions, are not meant to be limiting and may be implemented in various combinations in various aspects. Gene deletions may be accomplished by numerous strategies well known in the art, as are methods to incorporate foreign DNA into a host chromosome.

In various aspects, to function more efficiently, a microorganism may comprise one or more synthetic metabolic valves, comprised of enzymes targeted for controlled proteolysis, expression silencing, or a combination of both. For example, one enzyme encoded by one gene or a combination of numerous enzymes encoded by numerous genes in E. coli may be designed as synthetic metabolic valves to alter metabolism and improve product formation. Representative genes in E. coli may include but are not limited to the following: fabI, zwf, gltA, ppc, udhA, lpd, sucD, aceA, pfkA, lon, rpoS, pykA, pykF, tktA, and tktB. It is well known to one skilled in the art how to identify homologues of these genes and/or other genes in additional microbial species.

For all nucleic acid and amino acid sequences provided herein, it is appreciated that conservatively modified variants of these sequences are included and are within the scope of the invention in its various aspects. Functionally equivalent nucleic acid and amino acid sequences (functional variants), which may include conservatively modified variants as well as more extensively varied sequences, which are well within the skill of the person of ordinary skill in the art, and microorganisms comprising these, also are within the scope of various aspects of the invention, as are methods and systems comprising such sequences and/or microorganisms.

Accordingly, as described in various sections above, some compositions, methods and systems of the present invention comprise providing a genetically modified microorganism that comprises both a production pathway to make a desired product from a central intermediate in combination with synthetic metabolic valves to redistribute flux.

Aspects of the invention also regard provision of multiple genetic modifications to improve microorganism overall effectiveness in converting a selected carbon source into a selected product. Particular combinations are shown, such as in the Examples, to increase specific productivity, volumetric productivity, titer, and yield substantially over more basic combinations of genetic modifications.

In addition to the above-described genetic modifications, in various aspects genetic modifications, including synthetic metabolic valves, also are provided to increase the pool and availability of the cofactor NADPH and/or NADH, which may be consumed in the production of a product.

### V. Synthetic Metabolic Valves

Use of synthetic metabolic valves allows for simpler models of metabolic fluxes and physiological demands during a production phase, turning a growing cell into a stationary phase biocatalyst. These synthetic metabolic valves can be used to turn off essential genes and redirect carbon, electrons, and energy flux to product formation in a multi-stage fermentation process. One or more of the following provides the described synthetic valves: 1) transcriptional gene silencing or repression technologies in combination with 2) inducible and selective enzyme degradation and 3) nutrient limitation to induce a stationary or non-dividing cellular state. Synthetic metabolic valves are generalizable to any pathway and microbial host. Synthetic metabolic valves allow for novel rapid metabolic engineering strategies useful for the production of renewable chemicals and fuels and any product that can be produced via whole cell catalysis.

In particular, the invention describes the construction of synthetic metabolic valves comprising one or more or a combination of the following: controlled gene silencing and controlled proteolysis. One well skilled in the art is aware of several methodologies for gene silencing and controlled proteolysis.

### V.A Gene Silencing

In particular, the invention describes the use of controlled gene silencing to provide the control over metabolic fluxes in controlled multi-stage fermentation processes. There are several methodologies known in the art for controlled gene silencing, including but not limited to mRNA silencing or RNA interference, silencing via transcriptional repressors, and CRISPR interference. Methodologies and mechanisms for RNA interference are taught by Agrawal et al. “RNA Interference: Biology, Mechanism, and Applications” Microbiology and Molecular Biology Reviews, December 2003; 67 (4) p657-685. DOI: 10.1128/MMBR.67.657-685.2003. Methodologies and mechanisms for CRISRPR interference are taught by Qi et al. “Repurposing CRISPR as an RNA-guided platform for sequence-specific control of gene expression” Cell February 2013; 152 (5) p1173-1183. DOI: 10.1016/j.cell.2013.02.022. In addition, methodologies and mechanisms for CRISRPR interference using the native E. coli CASCADE system are taught by Luo et al. “Repurposing endogenous type I CRISPR-Cas systems for programmable gene repression” NAR. October 2014; DOI: 10.1093. In additional, numerous transcriptional repressor systems are well known in the art and can be used to turn off gene expression.

### V.B Controlled Proteolysis

In particular, the invention describes the use of controlled protein degradation or proteolysis to provide the control over metabolic fluxes in controlled multi-stage fermentation processes. There are several methodologies known in the art for controlled protein degradation, including but not limited to targeted protein cleavage by a specific protease and controlled targeting of proteins for degradation by specific peptide tags. Systems for the use of the E. coli clpXP protease for controlled protein degradation are taught by McGinness et al, “Engineering controllable protein degradation”, Mol Cell. June 2006; 22(5) p701-707. This methodology relies upon adding a specific C-terminal peptide tag such as a DAS4 (or DAS+4) tag. Proteins with this tag are not degraded by the clpXP protease until the specificity enhancing chaperone sspB is expressed. sspB induces degradation of DAS4 tagged proteins by the clpXP protease. In additional numerous site-specific protease systems are well known in the art. Proteins can be engineered to contain a specific target site of a given protease and then cleaved after the controlled expression of the protease. In some aspects, the cleavage can be expected lead to protein inactivation or degradation. For example, Schmidt et al (“ClpS is the recognition component for Escherichia coli substrates of the N-end rule degradation pathway” Molecular Microbiology March 2009. 72(2), 506-517. doi: 10.1111) teaches that an N-terminal sequence can be added to a protein of interest in providing clpS dependent clpAP degradation. In addition, this sequence can further be masked by an additional N-terminal sequence, which can be controllably cleaved by, e.g., a ULP hydrolase. This allows for controlled N-rule degradation dependent on hydrolase expression. It is therefore possible to tag proteins for controlled proteolysis either at the N-terminus or C-terminus. The preference of using an N-terminal vs. C-terminal tag will largely depend on whether either tag affects protein function prior to the controlled onset of degradation.

The invention describes the use of controlled protein degradation or proteolysis to provide the control over metabolic fluxes in controlled multi-stage fermentation processes, in E. coli. There are several methodologies known in the art for controlled protein degradation in other microbial hosts, including a wide range of gram-negative as well as gram-positive bacteria, yeast, and even archaea. In particular, systems for controlled proteolysis can be transferred from a native microbial host and used in a non-native host. For example, Grilly et al, “A synthetic gene network for tuning protein degradation in Saccharomyces cerevisiae” Molecular Systems Biology 3, Article 127. doi: 10.1038, teaches the expression and use of the E. coli clpXP protease in the yeast Saccharomyces cerevisiae. Such approaches can be used to transfer the methodology for synthetic metabolic valves to any genetically tractable host.

### V.C Synthetic Metabolic Valve Control

The invention describes the use of synthetic metabolic valves to control metabolic fluxes in multi-stage fermentation processes. There are numerous methodologies known in the art to induce expression that can be used at the transition between stages in multi-stage fermentations. These include but are not limited to artificial chemical inducers including: tetracycline, anhydrotetracycline, lactose, IPTG (isopropyl-beta-D-1-thiogalactopyranoside), arabinose, raffinose, tryptophan, and numerous others. Systems linking the use of these well-known inducers to the control of gene expression silencing and/or controlled proteolysis can be integrated into genetically modified microbial systems to control the transition between growth and production phases in multi-stage fermentation processes.

In addition, it may be desirable to control the transition between growth and production in multi-stage fermentations by the depletion of one or more limiting nutrients that are consumed during growth. Limiting nutrients can include but are not limited to: phosphate, nitrogen, sulfur, and magnesium. Natural gene expression systems that respond to these nutrient limitations can be used to operably link the control of gene expression silencing and/or controlled proteolysis to the transition between growth and production phases in multi-stage fermentation processes.

### VI. Ethanol as Carbon Source of Product Production

The invention describes chromosomal or plasmid modification of an endogenous adhE gene and an adaptation method in order to obtain a microorganism useful in a method of product production relying on ethanol as the carbon source for that product. The modification of the endogenous adhE gene is via a chromosomal modification and/or via a plasmid. In any event, the endogenous adhE is deleted or otherwise modified to render expression of the endogenous adhE gene impossible. The term endogenous refers to a native gene or in other words the copy of an adhE gene one would find in an unmodified microorganism. In some aspects, the endogenous adhE gene itself is deleted or otherwise modified, the promotor of an endogenous adhE gene is modified or both modification occur. In one aspect, the endogenous adhE gene promotor is modified to enable constitutive expression of a modified adhE gene from the chromosome. In some aspects the endogenous adhE gene is replaced with a mutant adhE gene. Such modification to the microorganism permit both growth and product production with ethanol as a carbon source. In some aspects, the growth phase occurs in a media comprising ethanol and in some aspects the growth phase occurs in a media comprising ethanol that is supplemented with glucose. The term growth media refers to media in which any microorganism is undergoing a growth phase. Consequently, production media is media in which a genetetically modified microorganism is found when it is undergoing or has undergone a transition from a growth phase to a product producing phase, and describes the media in which the microorgansims undergoes product production. In some aspects the product production phase occurs in a media where ethanol is the carbon source for the microorganism. In the inventive methods, ethanol serves as the carbon source for the product that is produced from the genetically modified microorganim according to aspects of the invention. Aspects of the invention describe use of the genetically modified microorganim in biofermentation methods.

### VII. Product Produced

There are no restrictions on the products that can be produced from ethanol as the carbon source as ethanol may be metabolized to acetyl-CoA, acetyl-CoA is then converted to pyruvate, from pyruvate cell growth and product formation occurs, as pyruvate is a key glycolysis intermediate. The product may comprise: an amino acid, acetate, acetoin, acetone, acrylic, malate, fatty acid ethyl esters, isoprenoids, terpene, glycerol, ethylene glycol, ethylene, propylene, butylene, isobutylene, ethyl acetate, vinyl acetate, 1,4-butanediol, 2,3-butanediol, butanol, isobutanol, sec-butanol, butyrate, isobutyrate, 2-OH-isobutryate, 3-OH-butyrate, ethanol, isopropanol, D-lactate, L-lactate, pyruvate, itaconate, levulinate, glucarate, glutarate, caprolactam, adipic acid, propanol, isopropanol, fused alcohols, 1,2-propanediol, 1,3-propanediol, formate, fumaric acid, propionic acid, succinic acid, valeric acid, maleic acid, poly-hydroxybutyrate, citramalate, or any citramalate derivative such as citraconic anhydride, itaconic acid, polyitaconate, itaconic polyester, polyol, a maleimide oligomer, a biscitraconimide monomer, protein linkage monomer, sodium sulfosuccinate esters, a maleic-plant oil derivative, or an alkenylsuccinic anhydride, or phloroglucinol.

### EXAMPLES

For the purposes of promoting an understanding of the principles of the present disclosure, reference will now be made to specific examples. No limitation of the scope of the claims is thereby intended.

### Example 1: Adaptation of Microorganism Strains to Ethanol as a Carbon Source

Two different microorganism strains had the endogenous adhE gene deleted (DMC_HS_044) and replaced by the adhE A267T/E568K mutant gene. The adhE A267T/E568K mutant gene expression product (i.e., the adhE A267T/E568K mutant polypeptide) allows E. Coli to use ethanol as the sole carbon source under aerobic conditions. The adhE A267T/E568K mutant gene may be supplied either in plasmid based format: (construct including DMC_PID_763; pCOLA backbone; Modified EM7 promoter (EM7*); Gentamicin resistance); or it can be supplied through integration into the E. Coli genome (DMC_HS_1944). The EM7-adhE A267T/E568K gene polynucleotide construct was integrated into DMC_HS_044 (F-, λ-, Δ(araD-araB) 567, lacZ4787 (del)(::rrnB-3), rph-1, Δ(rhaD-rhaB) 568, hsdR514, ΔackA-pta, ΔpoxB, ΔpflB, ΔldhA, EM7-adhE-A267T/E568K, ΔsspB, ΔiclR, ΔarcA, Δcas3::tm-ugpb-sspB-pro [casA*], gltA-das+4::zeoR, zwf-das+4::bsdR).

### Example 1A: Plasmid-Based adhE A267T/E568K Mutant Gene

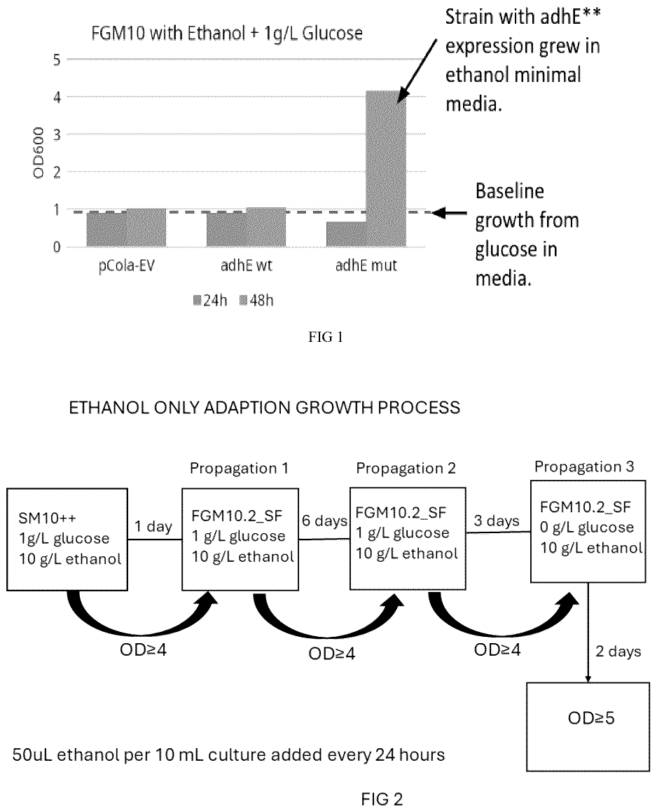

DMC_PID_763 was transformed using electroporation into DMC_HS_044 to create DMC_133. Transformants were cultivated at 30 C in DMC's rich media SM10++ supplemented with 1 g/L glucose and 10 g/L ethanol, and were propagated to DMC's minimal media FGM10.2_SF supplemented with 1 g/L glucose and 10 g/L ethanol for two passages. The adapted DMC_133 E. Coli strain was able to grow to higher OD. See FIG. 1.

### Example 1B: Chromosome-Based adhE A267T/E568K Mutant Gene

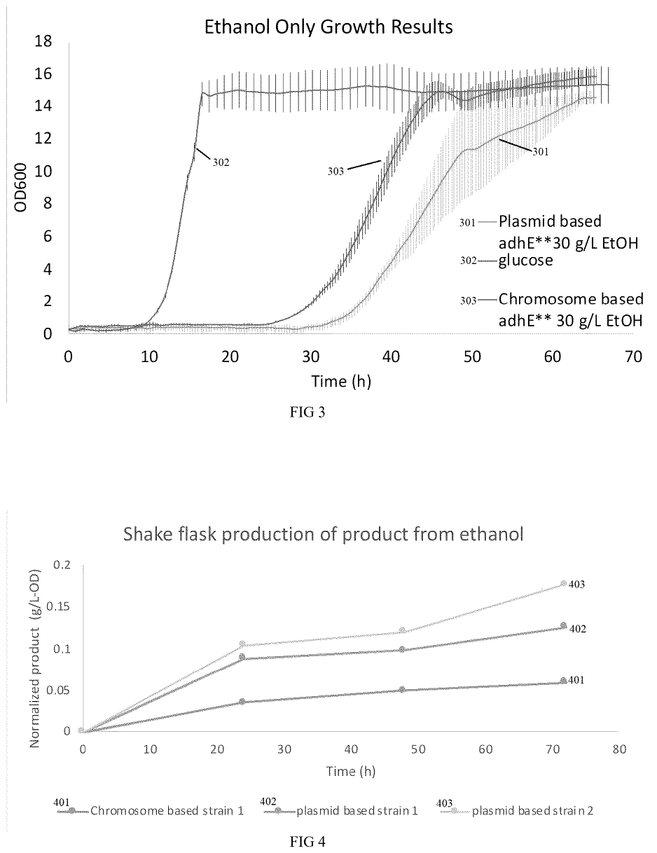

A single colony of DMC_HS_1944 was inoculated into DMC rich media (SM10++) supplemented with 1 g/L glucose and 10 g/L ethanol and cultivated at 30 C overnight (OD>4). The culture was used to inoculate (1%) 20 mL of DMC minimal media (FGM10.2_SF) supplemented with 1 g/L glucose and 10 g/L ethanol and cultivated at 30 C (propagation 1). Every 24 hours, the OD was measured, and 100 μL of 200 proof ethanol was added to the culture to account for the evaporation. Once the OD reached ~4 or more (~6 days), the culture was used to inoculate another flask of (FGM10.2_SF) supplemented with 1 g/L glucose and 10 g/L ethanol and cultivated at 30 C (propagation 2). Once the OD reached ~4 or more (~3 days), the culture was used to inoculate another flask of (FGM10.2_SF) supplemented with 0 g/L glucose and 10 g/L ethanol and cultivated at 30 C (propagation 3). This culture reached OD ~4 or more in 2 days and is now adapted to grow in ethanol only media. See FIG. 2.

### Example 1C: Transformation of Plasmids into Ethanol Adapted Strains

For either the plasmid-based or chromosome-based ethanol growth strains.

Grow cells to OD 0.5-1.0 in ethanol minimal media and make competent by washing 3× in 10% glycerol. Electroporate plasmid(s) into cells. Recover 3 hrs in 800 μL SM10++ 1 g/L glucose 10 g/L ethanol. Pellet cells.
Method 1: Resuspend in FGM10.2_SF 1 g/L glucose 10 g/L ethanol with antibiotics and inoculate 20 mL FGM10.2_SF 1 g/L glucose 10 g/L ethanol and begin ethanol only adaption growth process from first FGM10.2_SF step 1 g/L glucose 10 g/L ethanol. In some cases, the SM10++ recovery culture was added directly to FGM10.2_SF 1 g/L glucose 10 g/L ethanol.
Method 2: Resuspend in SM10++ 1 g/L glucose 10 g/L ethanol with antibiotics and inoculate 20 mL SM10++ 1 g/L glucose 10 g/L ethanol and begin ethanol only adaption growth process from SM10++ 1 g/L glucose 10 g/L ethanol step. In some cases, the SM10++ recovery culture was added directly to SM10++ 1 g/L glucose 10 g/L ethanol.

### Example 2: Growth Characterization of Ethanol Adapted Strains

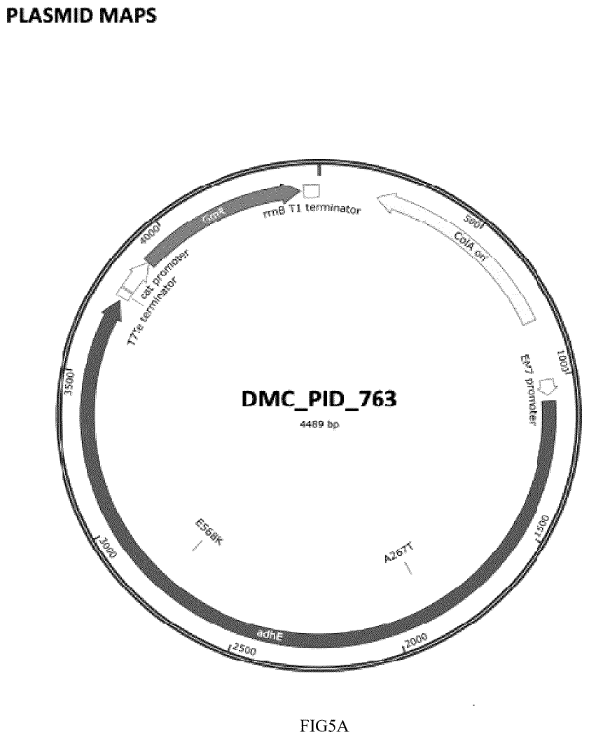

For either the plasmid-based or chromosome-based ethanol growth strain characterization was done at 30 and 37 C. OD data is collected using the BioLector. Prepare minimal media with 1 g/L glucose and 10, 20, and 30 g/L ethanol as well as no glucose and 10, 20, and 30 g/L ethanol. 760 μL of media+antibiotics in each well then, each well is inoculated with 40 μL of glycerol stock. 30 and 37 C 1300 rpm runs for several days. See FIG. 3.

### Example 3: Production of a Desired Product

The same protocol is used for both chromosomal and plasmid-based ethanol growth strains. Starting from propagation 3 in the adaption process (media FGM10.2_SF 0 g/L glucose and 10 g/L ethanol) the strains were grown until OD>5. 4 OD of cells were removed from the flask, washed twice with production media and then brought to a final volume of 10-20 mL in a shake flask. The flask with 4 OD of cells in production media was then incubated at 30 or 37 C for 72 hours with time points being taken every 24 hours. Ethanol (50 μL for every 10 mL of media) was added every 24 hours to the shake flasks to account for evaporation. To take the timepoints, 1 mL of culture was removed and transferred to microtube. The OD was measured, and the cells were pelleted by centrifugation and the supernatant was measured for the production of the desired final product in triplicate for each timepoint. This procedure was demonstrated for multiple products including citramalate and phloroglucinol.

1. A multi-stage biofermentation process for producing a product from a genetically modified microorganism, comprising:
(A) providing a genetically modified microorganism that:
(1) is adapted to grow in a growth media comprising ethanol; and
(2) comprises:
(a) a production pathway comprising at least one production enzyme for biosynthesis of the product;
(b) a mutation of the endogenous adhE gene and of the endogenous adhE gene promotor; and
(c) one or more synthetic metabolic valves for reducing or eliminating flux through multiple metabolic pathways within the genetically modified microorganism when the synthetic metabolic valves are induced;
(B) growing the genetically modified microorganism in a media comprising ethanol in a growth phase; and
(C) transitioning to a productive stationary phase, the transitioning comprising:
(1) depleting a limiting nutrient;
(2) inducing the one or more synthetic metabolic valves; and
(3) activating the production pathway,
wherein the growth or production media comprising ethanol comprises at least 10 g/L ethanol and no added glucose.

2. The method of claim 1, wherein the mutation of the endogenous adhE gene comprises chromosomal deletion of the endogenous adhE gene and replacement with adhE A267T/E568K mutant gene.

3. The method of claim 1, wherein the mutation of the endogenous adhE gene comprises chromosomal deletion of the endogenous adhE gene and transformation of the microorganism with a plasmid encoding A267T/E568K adhE mutant gene.

4. (canceled)

5. The method of claim 2, wherein the endogenous adhE gene promotor is replaced by a constitutive promotor for constitutive expression of the adhE mutant gene.

6. The method of claim 1, wherein the one or more synthetic metabolic valves comprises: a) at least one silencing synthetic metabolic valve that silences gene expression of a gene selected from: fabI, gltA, lpd, zwf, and udhA, or b) at least one proteolytic synthetic metabolic valve that controls proteolysis of a proteolyzable enzyme selected from: fabI, gltA, lpd, zwf, and udhA.

7. The method of claim 1, wherein the product comprises: an amino acid, acetate, acetoin, acetone, acrylic, malate, fatty acid ethyl esters, isoprenoids, glycerol, ethylene glycol, ethylene, propylene, butylene, isobutylene, ethyl acetate, vinyl acetate, 1,4-butanediol, 2,3-butanediol, butanol, isobutanol, sec-butanol, butyrate, isobutyrate, 2-OH-isobutryate, 3-OH-butyrate, ethanol, isopropanol, D-lactate, L-lactate, pyruvate, itaconate, levulinate, glucarate, glutarate, caprolactam, adipic acid, propanol, isopropanol, fused alcohols, 1,2-propanediol, 1,3-propanediol, formate, fumaric acid, propionic acid, succinic acid, valeric acid, maleic acid, poly-hydroxybutyrate, citramalate or phloroglucinol.

8. A method of adapting a genetically modified E. coli microorganism comprising synthetic metabolic valves to grow aerobically in a growth medium comprising ethanol, the method comprising:
providing a microorganism with a deletion of endogenous adhE gene, a mutation of the endogenous adhE gene promotor, and replacement with adhE A267T/E568K mutant gene and a constitutive promotor;
growing the microorganism in an ethanol minimal media;
modifying the microorgansism with plasmids encoding synthetic metabolic valves; and
propagating the modified microorganism in a growth process in a media comprising SM10++ or FGM10.2_SF 1 g/L glucose and 10 g/L ethanol for at least three propagations.

9. The method of claim 8, the genetically modified E. coli further comprising chromosomal modifications that complement the portion of the synthetic metabolic valve encoded by the plasmid.

10. The method of claim 9, wherein the chromosomal modification of the genetically modified E. coli includes introduction into the chromosome of a proteolytic synthetic metabolic valve that controls proteolysis of an enzyme.

11. A genetically modified E. coli microorganism comprising a production pathway comprising:
at least one production enzyme for biosynthesis of the product;
a mutation of the endogenous adhE gene and a mutation of the endogenous adhE gene promotor; and
one or more synthetic metabolic valves for reducing or eliminating flux through multiple metabolic pathways within the genetically modified microorganism when the synthetic metabolic valves are induced.

12. The microorganism of claim 11, wherein the mutation of the endogenous adhE gene comprises chromosomal deletion of an endogenous adhE gene and replacement of endogenous adhE gene with adhE A267T/E568K mutant gene.

13. The microorganism of claim 11, wherein the mutation of the endogenous adhE gene comprises chromosomal deletion of an endogenous adhE gene and transformation of the microorganism with a plasmid encoding adhE A267T/E568K mutant gene.

14. The method of claim 12, wherein the endogenous adhE gene promotor is replaced by a constitutive promotor for constitutive expression of the adhE mutant gene

15. The microorganism of claim 11, wherein the microorganism grows in a growth or production media comprising ethanol at a concentration of at least 10 g/L ethanol and no glucose.

16. The microorganism of claim 11, wherein the one or more synthetic metabolic valves comprises: a) at least one silencing synthetic metabolic valve that silences gene expression of a gene selected from: fabI, gltA, lpd, zwf, and udhA, or b) at least one proteolytic synthetic metabolic valve that controls proteolysis of a proteolyzable enzyme selected from: fabI, gltA, lpd, zwf, and udhA.


SEQUENCE LISTING (4 sequences)

SEQ ID NO: 1 - DNA, 66 bp, synthetic construct
  source 1..66: mol_type = other DNA
ggtttagttcctcaccttgtcgtattatactatgccgatatactatgccgatgattaattgtcaac

SEQ ID NO: 2 - DNA, 35 bp, synthetic construct
  source 1..35: mol_type = other DNA
gttgacaattaatcatcggcatagtataatacgac

SEQ ID NO: 3 - DNA, 2676 bp, synthetic construct
  source 1..2676: mol_type = other DNA
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttaccacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaaaaactggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 4 - protein, 891 aa, synthetic construct
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFTTHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEKLALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA